SEQUENCE LISTING (8 sequences)

SEQ ID NO: 1 - protein, 429 aa, Homo sapiens
MASNSIFDSFPTYSPTFIRDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPY

SEQ ID NO: 2 - protein, 415 aa, Homo sapiens
MRIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPY

SEQ ID NO: 3 - DNA, 1290 bp, Homo sapiens
  source 1..1290: mol_type = other DNA
atggcatcgaacagcatcttcgactccttcccgacctactcgccgaccttcatccgcgacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctactga

SEQ ID NO: 4 - DNA, 1248 bp, Homo sapiens
  source 1..1248: mol_type = other DNA
atgcgtattcccgtagacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctactga

SEQ ID NO: 5 - DNA, 9106 bp, synthetic construct
  source 1..9106: mol_type = other DNA
  misc_feature 1..9106: note = FRT STOP FRT cassette
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatcaagcttggtaccgagctcggatccgtcgacgtttgaagttcctatactttctagagaataggaacttcgtttctcgagctgcagacttactcaagccatggtggctagcttgcggaattccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccctcgaccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccctcgaccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccctcgaccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccctcgatcgagaaaaaaaatataaaagagatggaggaacgggaaaaagttagttgtggtgataggtggcaagtggtattccgtaagaacaacaagaaaagcatttcatattatggctgaactgagcgaacaagtgcaaaatttaagcatcaacgacaacaacgagaatggttatgttcctcctcacttaagaggaaaaccaagaagtgccagaaataacatgagcaactacaataacaacaacggcggctacaacggtggccgtggcggtggcagcttctttagcaacaaccgtcgtggtggttacggcaacggtggtttcttcggtggaaacaacggtggcagcagatctaacggccgttctggtggtagatggatcgatggcaaacatgtcccagctccaagaaacgaaaaggccgagatcgccatatttggtgtccccgaggatccctgtggaaaaaaaagggacaggataagtatgacatcatcaaggaaaccctggactactgcgcccttcgacctcgaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcatccatctagatctcgatcgagcagctgaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgatgatatcagatccccgggatgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtaacggctcgaggtttgaagttcctatactttctagagaataggaacttcgttgtcgacgcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 6 - DNA, 21 bp, synthetic construct
  source 1..21: mol_type = other DNA
  misc_feature 1..21: note = Runx3 SC F1 forward A
ctgtgtagtcctggctatcct

SEQ ID NO: 7 - DNA, 21 bp, synthetic construct
  source 1..21: mol_type = other DNA
  misc_feature 1..21: note = Runx3 SC R1 reverse B
cttagctgtcctccgactaca

SEQ ID NO: 8 - DNA, 21 bp, synthetic construct
  source 1..21: mol_type = other DNA
  misc_feature 1..21: note = OS Neo F1 reverse C
ggatgatctggacgaagagca

(19) United States
(12) Patent Application Publication
(10) Pub. No.: US 2026/0130967 A1
(43) Pub. Date: May 14, 2026

(54) PHARMACEUTICAL COMPOSITION COMPRISING RUNX3 GENE OR PROTEIN AS ACTIVE INGREDIENT FOR PREVENTION OR TREATMENT OF K-RAS MUTANT LUNG CANCER

(71) Applicant: BIORUNX, Cheongju-si (KR)

(72) Inventors: Suk-Chul Bae, Cheongju-si (KR); You-Soub Lee, Cheongju-si (KR); Ja-Yeol Lee, Cheongju-si (KR)

(73) Assignee: BIORUNX, Cheongju-si (KR)

(21) Appl. No.: 19/326,473

(22) Filed: Sep. 11, 2025

Related U.S. Application Data
(63) Continuation of application No. 17/421,550, now abandoned.

(30) Foreign Application Priority Data
Jan. 8, 2019 (KR) 10-2019-0002242

(51) Int. Cl. A61K 38/17; A61K 49/00; A61P 35/00

(52) U.S. Cl. CPC A61K 38/1709; A61K 49/0004; A61P 35/00

(57) ABSTRACT

The present invention relates to a pharmaceutical composition comprising a Runx3 gene or protein as an active ingredient for prevention or treatment of K-Ras mutant lung cancer. Specifically, Runx3 gene-deleted, K-Ras gene-activated lung cancer mice established in the present invention were found to be completely cured without lung cancer recurrence likelihood when restoring the Runx3 gene, compared to the conventional approach of inhibiting the activated cancer gene. Thus, the composition comprising Runx3 protein, a polynucleotide coding therefor, a vector carrying the polynucleotide, or a virus or cell transformed with the vector as an active ingredient according to the present invention can be advantageously used as a composition for prevention or treatment of K-Ras mutant lung cancer.

### CROSS REFERENCE TO RELATED APPLICATIONS

This application is a continuation of U.S. patent application Ser. No. 17/421,550, filed Jul. 8, 2021, which is the U.S. National Stage of International Application No. PCT/KR2019/008629, filed Jul. 12, 2019, which in turn claims the benefit of priority from Korean Patent Application No. 10-2019-0002242, filed Jan. 8, 2019, the contents of each of which are incorporated herein by reference.

### INCORPORATION OF ELECTRONIC SEQUENCE LISTING

The Sequence Listing is submitted as an XML file in the form of the file named “Sequence.xml” (21,893 bytes), which was created on Sep. 10, 2025, and which is incorporated by reference herein.

### BACKGROUND OF THE INVENTION

### 1. Field of the Invention

The present invention relates to a pharmaceutical composition comprising a Runx3 (Runt-related transcription factor 3) gene or protein as an active ingredient for prevention or treatment of K-Ras mutant lung cancer.

### 2. Description of the Related Art

Lung cancer deaths account for the highest proportion of cancer deaths, and about 1.3 million people worldwide die of lung cancer every year. Lung cancer is classified as small cell lung cancer when the size of the cancer cell is small, and non-small cell lung cancer when the size of the cancer cell is not small. Among them, small cell lung cancer accounts for about 15% of all lung cancers, and it is common among smokers. It is an aggressive form of cancer and has a low survival rate. Non-small cell lung cancer is further divided into squamous cell carcinoma, large cell carcinoma, and lung adenocarcinoma. Lung adenocarcinoma is a cancer that occurs in the glandular tissues of the lungs, that is, small peripheral bronchial epithelium, which are cells that secrete body fluids. This is common in non-smokers and women, and it is often metastasized even if the size is small. Lung adenocarcinoma is known to account for about 35-40% of lung cancer.

Research on the development of targeted cancer therapy is focused on strategies to control cancer cells by inhibiting the function of an oncogene or activating the function of a tumor suppressor gene. Abnormal activation of K-Ras function by mutation of K-Ras among the oncogenes is known as one of the major causes of human cancer. The mutation of K-Ras is also observed in lung cancer, and it is known that the mutation of K-Ras is observed in about 35% of lung adenocarcinoma. Thus, in order to treat cancer caused by the activation of K-Ras function, studies have been conducted on a method of treating cancer by inhibiting the function of K-Ras. However, a strategy that directly inhibits the function of K-Ras has not been developed as a successful anticancer drug because it causes serious damage to normal cells. Therefore, instead of suppressing the function of an oncogene, a strategy of activating the inhibited function of a tumor suppressor gene is receiving attention. Therefore, instead of a strategy for inhibiting the function of an oncogene, a strategy for activating the inhibited function of a tumor suppressor gene is attracting attention.

The said tumor suppressor gene refers to a nucleotide sequence that can be expressed in a target cell to suppress a tumor phenotype or induce apoptosis. The tumor suppressor genes identified so far include sPD-1, VHL, MMACI, DCC, p53, NF1, WT1, Rb, BRCA1 and BRCA2. Among them, it has been reported that p53 or Rb gene is frequently inhibited in its function in K-Ras mutant cancers. Whether it is possible to treat K-Ras mutant cancer through the repair of the suppressor gene has become a subject of great interest in the field of anticancer agent development research. Accordingly, there have been attempts to treat K-Ras mutant lung adenocarcinoma by recovering the function of p53 gene, which is a representative tumor suppressor gene, but it was not successful because early lung adenocarcinoma was not cured (Feldser, D. M. et al., Nature, 468:572-575, 2010, Junttila, M. R. et al., Nature, 468:567-571, 2010). In addition, it was found that K-Ras mutant lung cancer could not be cured through the recovery of Rb gene function (Walter, D. M. et al. Nature 2019). The above results indicate that even if the function of the tumor suppressor gene is simply restored, the therapeutic effect on the already-onset cancer does not appear, because the early stage cancer rapidly develops into a malignant cancer (Berns A., Nature, 468:519-520, 2010). There have been no reports of successful treatment of K-Ras mutant lung cancer through the activation of a tumor suppressor gene.

The above results indicate that even if a gene identified as a cancer suppressor gene is suppressed in a specific cancer, whether or not a cancer treatment effect will appear in an actual animal through the activation of the cancer suppressor gene cannot be predicted without direct animal experiments. Therefore, a cancer treatment strategy through the activation of a tumor suppressor gene cannot be a successful cancer treatment strategy unless a specific cancer with a specific condition and a tumor suppressor gene cannot be selected.

As Runx3 gene was found to be a tumor suppressor gene, it was expected that cancer treatment effect through the activation of Runx3 gene was expected, but there is no report that it actually has cancer treatment effect in animal models. Rather, it has been reported that Runx3 gene acts as an oncogene depending on the type of cancer (Lee et al., Gynecol. Oncol., 122 (2): 410-417, 2011, Kudo Y. et al., J. Cell Biochem., 112 (2): 387-393, 2011).

It has been reported that the function of Runx3 gene as a tumor suppressor gene is inhibited in K-Ras mutant cancers (RUNX3 Protects against Oncogenic KRAS. (2013). Cancer Discovery, 4 (1), 14-14), and that the activity of Runx3 gene is inhibited in lung adenocarcinoma caused by the mutation of K-Ras (Lee, K. S., Lee, Y. S., Lee, J. M., Ito, K., Cinghu, S., Kim, J. H., Bae, S. C. Oncogene, 29 (23): 3349-61, 2010.).

However, as can be seen from the case of p53, whether or not a cancer treatment effect will appear by enhancing the activity of Runx3 gene in K-Ras mutant lung cancer cannot be predicted without direct experiments.

Thus, the present inventors confirmed that lung cancer develops only when K-Ras gene is activated and Runx3 gene is suppressed, and completed the present invention by confirming in an animal cancer model that lung cancer is treated when Runx3 gene is activated and Runx3 is expressed in K-Ras mutant lung cancer.

### SUMMARY OF THE INVENTION

It is an object of the present invention to provide a composition for prevention or treatment of K-Ras mutant lung cancer, comprising a Runx3 protein, a polynucleotide coding thereof, a vector carrying the polynucleotide, or a virus or cell transformed with the vector as an active ingredient.

It is another object of the present invention to provide a screening method of a candidate substance for treating K-Ras mutant lung cancer.

It is another object of the present invention to provide a method for preventing, ameliorating or treating K-Ras mutant lung cancer comprising a step of administering the Runx3 (Runt-related transcription factor 3) protein, the polynucleotide coding thereof, the vector carrying the polynucleotide, or the virus or cell transformed with the vector to a subject.

It is another object of the present invention to provide a use of the Runx3 (Runt-related transcription factor 3) protein, the polynucleotide coding thereof, the vector carrying the polynucleotide, or the virus or cell transformed with the vector for the manufacture of a medicament for preventing, ameliorating or treating K-Ras mutant lung cancer.

To achieve the above objects, the present invention provides a pharmaceutical composition for prevention or treatment of K-Ras mutant lung cancer, comprising a Runx3 protein, a polynucleotide coding thereof, a vector carrying the polynucleotide, or a virus or cell transformed with the vector as an active ingredient.

The present invention also provides a screening method of a candidate substance for treating K-Ras mutant lung cancer comprising the following steps:
1) treating a test substance to the cells containing Runx3 gene;
2) confirming the expression or activity of Runx3 protein in the cells of step 1); and
3) selecting a test substance that increases the expression or activity of Runx3 protein in step 2) compared to the untreated control group.

The present invention also provides a method for preventing, ameliorating or treating K-Ras mutant lung cancer comprising a step of administering the Runx3 (Runt-related transcription factor 3) protein, the polynucleotide coding thereof, the vector carrying the polynucleotide, or the virus or cell transformed with the vector to a subject.

In addition, the present invention provides a use of the Runx3 (Runt-related transcription factor 3) protein, the polynucleotide coding thereof, the vector carrying the polynucleotide, or the virus or cell transformed with the vector for the manufacture of a medicament for preventing, ameliorating or treating K-Ras mutant lung cancer.

### Advantageous Effect

When the Runx3 activity is restored in lung cancer caused by activation of K-Ras mutant gene and decrease in the activity of Runx3 protein, the lung cancer cells are removed and the normal cells survive. Therefore, lung cancer can be fundamentally cured by administering a Runx3 protein, a polynucleotide coding therefor, a vector carrying the polynucleotide, or a virus or cell transformed with the vector.

### BRIEF DESCRIPTION OF THE DRAWINGS

FIG. 1a is a diagram illustrating the survival of the mice produced (K-Ras^{LSL-G12D}; Runx3^{Flox} normal mice (KR) without Cre^{tm/ERT1} in which K-Ras gene is inactivated and Runx3 gene is activated, K-Ras^{LSL-G12D}; Cre^{tm/ERT1} mice (K-Cre^{ERT1}) with Cre^{tm/ERT1} in which K-Ras gene is activated in very few cells, K-Ras^{LSL-G12D}; p53flox; Cre^{tm/ERT1} mice (KP-Cre^{ERT1}) in which K-Ras gene is activated in very few cells and p53 gene is suppressed, Runx3Flor, Crem ERT¹ mice (R-Cre^{ERT1}) in which Runx3 gene is suppressed in very few cells, and K-Ras^{LSL-G 12D}; Runx3^{Flox}; Cre^{tm/ERT1} mice (KR-Cre^{ERT1}) in which K-Ras gene is activated in very few cells and Runx3 gene is suppressed), confirming that KR mice, K-Cre^{ERT1} mice, KP-Cre^{ERT1} mice, and R-Cre^{ERT1} mice survived healthy for more than 1 year, but KR-Cre^{ERT1} mice all died within 85 days of birth, and that fatal disease occurred only in mice in which K-Ras mutation occurred and Runx3 was suppressed.

FIG. 1b is a photograph showing the lung tissues after raising the K-Cre^{ERT1} mice, KP-Cre^{ERT1} mice and R-Cre^{ERT1} mice of FIG. 1, confirming that the healthy lung tissues such as those of normal mice were observed in K-Cre^{ERT1} mice and KP-Cre^{ERT1} mice, and dysplasia was observed very rarely in R-Cre^{ERT1}, but cancer did not develop, and that cancer did not develop with the K-Ras mutant cancer gene alone, and the destruction of p53 did not promote the onset of cancer by the K-Ras mutant cancer gene.

FIG. 1c is a photomicrograph showing that lung adenocarcinoma began to be observed from 2 weeks after birth in the KR-Cre^{ERT1} mice of FIG. 1a, confirming that lung cancer developed only when K-Ras oncogene mutation occurred in the cells in which Runx3 was destroyed, and that Runx3 strongly inhibited the development of lung cancer caused by K-Ras oncogene mutation.

FIG. 1d is a diagram showing the lung tissues of K-Ras^{LSL-G12D}, p53^{flox}, R26T;Cre^{tm/ERT2} mice (KPT-Cre^{ERT2}) in which K-Ras gene is activated in very few cells and p53 gene is suppressed by Cre^{tm/ERT1} and K-Ras^{LSL-G12D}; Runx3^{Flox}; R26T; Cre^{tm/ER12} mice (KRT-Cre^{ERT2}) in which K-Ras gene is activated in very few cells and Runx3 gene is suppressed grown for 6 months in the absence of tamoxifen, which were stained with H&E and observed under a microscope, confirming that lung cancer did not occur at all in KPT-Cre^{ERT2} mice, but a number of lung adenocarcinomas occurred in KRT-Cre^{ERT2} mice, and reconfirming that the gene that induced the development of lung cancer caused by K-Ras oncogene mutation was not p53 but Runx3

FIG. 1e is a diagram showing the lung tissues of K-Ras^{LSL-G12D}; R26T; Cre^{tm/ERT2} mice (KT-Cre^{ERT2}) in which K-Ras gene is activated in very few cells, which were stained with anti-tomato antibody and observed under a microscope, confirming that the cells obtained the designed genetic mutation and hardly divide by identifying the tomato-positive red cells indicated by arrows.

FIG. 1f is a diagram showing the lung tissues of K-Ras^{LSL-G12D};p53^{flox}. R26T;Cre^{tm/ERT2} mice (KPT-Cre^{ERT2}) in which K-Ras gene is activated in very few cells and p53 gene is suppressed, which were stained with H&E and anti-tomato antibody and observed under a microscope, confirming that cancer was not formed with normal tissues although a large number of tomato-positive cells were observed, and confirming that the deletion of p53 gene and the mutation of K-Ras gene were not sufficient conditions for the onset of cancer. That is, it was found that p53 did not inhibit the occurrence of lung cancer caused by the mutation in K-Ras oncogene.

FIG. 1g is a diagram showing the lung tissues of K-Ras^{LSL-G1 2D};Runx3^{Flox}; R26T;Cre^{tm/ERT2} mice (KRT-Cre^{ERT2}) in which K-Ras gene is activated in very few cells and Runx3 gene is suppressed, which were stained with H&E and anti-tomato antibody and observed under a microscope, confirming that there were many tomato-positive cells, forming lung cancer, and that the deletion of Runx3 gene and the mutation of K-Ras oncogene were sufficient conditions for the onset of cancer. That is, it was found that Runx3 was a gene that suppressed the occurrence of lung cancer caused by the mutation of K-Ras oncogene.

FIG. 2 is a diagram showing the genetic map in which the FRT-STOP-FRT cassette (5492 bp) is inserted into the SphI restriction enzyme site located at the 5′-intron of exon 2 and exon 3 of Runx3 gene. The FRT-STOP-FRT cassette can be removed by Flippase DNA recombinase.

FIGS. 3a to 3d are diagrams showing the nucleotide sequence of the vector (FRT-STOP-FRT TOPO plasmid, Cat #. 22774) including the FRT-STOP-FRT cassette (underlined part: FRT sequence). The nucleotide sequence shown is SEQ ID NO: 5.

FIG. 4 is a diagram showing the results of Southern blotting in which gDNA was extracted from the FRT-STOP-FRT cassette introduced gene targeted embryonic stem cells, and the transformed embryonic stem cells were selected using the 5′-probe shown in FIG. 1.

FIG. 5 is a diagram showing the results of polymerase chain reaction (PCR) using a primer capable of complementary binding to the regions indicated by A, B and C of FIG. 1 to select Runx3^{FRT-STOP-FRT} knock-in mice. In the case of a mouse to which a target gene (FRT-STOP-FRT cassette) was successfully introduced, a PCR reaction occurs by a primer that complementarily binds to A and C sites, and in the case of a mouse to which a target gene was not introduced, a PCR reaction occurs by a primer that complementarily binds to A and B sites. Therefore, knock-in mice can be selected. As a result of electrophoresis with the obtained PCR product, a group in which a band was formed at 542 bp (the group marked with *) was finally selected as Runx3^{FRT-STOP-FRT} knock-in mice.

FIG. 6a is a diagram showing the genetic map of K-Ras^{LSL-G12D} (A) capable of selectively activating K-Ras gene by removing Stop sequence when the Cre recombinant enzyme is introduced.

FIG. 6b is a diagram showing the genetic map of Runx3^{Flox} (B) capable of selectively inhibiting the expression of Runx3 gene by removing exon 4 sequence of Runx3 gene when the Cre recombinant enzyme is introduced.

FIG. 6c is a diagram showing the genetic map of R26^{FlpoER} (C) capable of introducing the Flippase fused with an estrogen receptor into the nucleus during the treatment with tamoxifen, an estrogen analogue.

FIG. 6d is a diagram showing the genetic map of R26T (D) capable of selectively expressing tdTomato, a red fluorescent protein, by removing Stop sequence when the Cre recombinase is introduced.

FIG. 7 is a diagram showing the mating process for producing Runx3^{Flox}/FRT-STRP-FRT.K_Ras^{LSL-G12D}: R26^{FlpoER};R26T mice.

FIG. 8 is a photograph showing the tdTomato expression in the lung cancer cells formed by lung cancer as a result of respiratory infection of the produced Runx3^{Flox/FRT-STRP-FRT}; K-Ras^{LSL-G12D}; R26^{FlpoER}; R26T mice with adenovirus expressing the Cre recombinase to activate K-Ras gene and suppress the expression of Runx3 gene (DAPI: staining the cell nucleus).

FIG. 9 is a schematic diagram showing the genetic change process in the case of developing lung cancer by activating K-Ras gene by respiratory infecting the produced Runx3^{Flox/FRT-STRP-FRT}; K-Ras^{LSL-G}12D, R26TlpoER; R26T mice with Cre-adenovirus and inhibiting the expression of Runx3 gene and in the case of restoring Runx3 gene by introducing Flippase into the nucleus by the administration of tamoxifen.

FIG. 10 is a photograph of the lung tissues extracted from the control mouse (Control) in which lung adenocarcinoma was developed by activating K-Ras gene and suppressing the expression of Runx3 gene and the mouse in which Runx3 gene was restored.

FIG. 11 is a photograph showing the lung tissues extracted from the control mouse (Control) in which lung adenocarcinoma was developed by activating K-Ras gene and suppressing the expression of Runx3 gene and the mouse in which Runx3 gene was restored, which were stained with H&E (Hematoxilin & Eosin), confirming that lung adenocarcinoma was almost eliminated from the lung tissue of the mouse group in which Runx3 gene was restored.

FIG. 12 is an enlarged view of a part among the lung tissues of #2 mouse of the Runx3 restored group of FIGS. 9 and 10 that looked somewhat abnormal, confirming that this part was not the abnormal tissue as a sign of cancer formation and treatment.

FIG. 13a is a diagram illustrating the survival of the control mice in which lung adenocarcinoma was induced by infecting Runx3floxFSF; K-Ras^{LSL-G12D},Flp-ERT2 mice with Cre-adenovirus to induce K-Ras mutation and Runx3 inactivation and fed with tamoxifen-free diet, and the mice in which Runx3 was restored by feeding feed containing tamoxifen from 6 weeks after the Cre-adenovirus infection. The mice in which Runx3 was not restored by feeding tamoxifen-free diet died within 14 weeks after the infection with Cre-adenovirus However, the control mice fed with tamoxifen-containing diet (Runx3 restored mice) survived until 24 weeks after the Cre-adenovirus infection. The above results indicate that the survival rate of lung adenocarcinoma was significantly increased by restoring Runx3.

FIG. 13b is a schematic diagram showing the control mice (ctrl-6w) that were sacrificed 6 weeks after infecting Runx3^{flox} FSF, K-Ras^{LSL-G12D};Flp-ERT2 mice with Cre-adenovirus and feeding with tamoxifen-free diet, the control mice (ctrl-10w) that were sacrificed 10 weeks after infecting with Cre-adenovirus and feeding with tamoxifen-free diet, and the Runx3 restored group mice (tam-10w) fed with tamoxifen-containing diet for 4 weeks after 6 weeks of the Cre-adenovirus infection.

FIG. 13c is a photograph showing the lung tissues extracted from the control mice (ctrl-6w) that were sacrificed 6 weeks after infecting Runx3^{flox/FSF}; K-Ras^{LSL-G12D}, Flp-ERT2 mice with Cre-adenovirus and feeding with tamoxifen-free diet, the control mice (ctrl-10w) that were sacrificed 10 weeks after infecting with Cre-adenovirus and feeding with tamoxifen-free diet, and the Runx3 restored group mice (tam-10w) fed with tamoxifen-containing diet for 4 weeks after 6 weeks of the Cre-adenovirus infection, which were stained with H&E (Hematoxilin & Eosin), confirming that almost all lung adenocarcinoma was removed by restoring Runx3.

FIG. 14a is a schematic diagram showing the control mice (ctrl-T*-6w) that were sacrificed 6 weeks after infecting Runx3^{Flox}/FRT-STOP-FRT;K-Ras^{LSL-G12D}; R26^{FlpoER}; R26T mice with Cre-adenovirus and feeding with tamoxifen-free diet, the control mice (ctrl-T*-10w) that were sacrificed 10 weeks after infecting with Cre-adenovirus and feeding with tamoxifen-free diet, and the Runx3 restored group mice (tam-T*-16w) fed with tamoxifen-containing diet for 10 weeks after 6 weeks of the Cre-adenovirus infection.

FIG. 14b is a photograph illustrating the tdTomato protein fluorescence of the lung tissues of the ctrl-T*-6w, ctrl-T*-10w and tam-T*-16w of FIG. 14a entirely taken under ultraviolet light, confirming that ctrl-T*-6w and ctrl-T*-10w mice showed fluorescence and thus lung cancer was developed, and that tam-T*-16w mice in which Runx3 was restored after the onset of lung adenocarcinoma showed little fluorescence, so lung cancer was treated.

FIG. 14C is a set of photomicrographs showing the lung tissues extracted from ctrl-T*-6w, ctrl-T*-10w and tam-T*-16w mice, which were stained with H&E (Hematoxilin & Eosin) (left) and anti-tomato antibody (right), confirming that large-sized lung cancer was found in ctrl-T*-6w and ctrl-T*-10w mice, but lung cancer was not observed in tam-T*-16w mice, so that the generated lung cancer was removed by restoring Runx3.

FIG. 15 is an enlarged diagram of FIG. 13c showing TUNEL (Terminal deoxynucleotidyl transferase dUTP nick-end labeling) staining for confirming the dead cells and DAPI (4′,6-diamidino-2-phenylindole) staining for staining the cell nucleus, confirming that live lung cancer filling the lung tissue was observed in ctrl-6w and ctrl-10w, and the lung cancer was disappeared in tam-10w, and that the part that looked somewhat abnormal was the cells that had already died as a result of TUNEL (Terminal deoxynucleotidyl transferase dUTP nick-end labeling) staining, and this part was the normal lung tissue as a result of DAPI staining. The above results indicate that the lung cancer that had already been created was eliminated by restoring Runx3.

FIG. 16 is an enlarged diagram of FIG. 14c, confirming that the lung cancer that had already been created was completely removed by restoring Runx3 since the part of the lung tissue of tam-T*-16w that looked somewhat abnormal was normal alveoli.

### DESCRIPTION OF THE PREFERRED EMBODIMENTS

Hereinafter, the present invention is described in detail.

The present invention provides a pharmaceutical composition for prevention or treatment of K-Ras mutant lung cancer, comprising a Runx3 (Runt-related transcription factor 3) protein, a polynucleotide coding thereof, a vector carrying the polynucleotide, or a virus or cell transformed with the vector as an active ingredient.

Runx3 (Runt-related transcription factor 3) gene is one of the Runt family genes consisting of Runx1, Runx2 and Runx3. The Runt family genes play an important role in normal development and oncogenesis, and they function as transcriptional regulators of the Smad family, a downstream factor that mediates TGF-B and its signaling. Runx1 plays an important role in mammalian hematopoiesis, Runx2 plays an important role in bone formation, and Runx3 is mainly expressed in granular gastric mucosal cells, and plays a role in inhibiting cell differentiation of gastric epithelium. These three genes are located at loci of chromosomes 1p, 6p and 21q, of which Runx3 gene is located at 1p36. 11-1p36. 13. The Runx3 locus is one of the sites that are lost in a variety of cancers or affected by hemizygous defects. In addition, Runx.3 has been found to be inactivated in various types of cancer, and it is gaining spotlight as a new target for the development of anticancer agents As such, Runx3 is known not only to act as a tumor suppressor gene that suppresses the formation of cancer, but also to suppress cancer metastasis. Runx3 plays an important role in the restriction-point, which determines the fate of cell division and death, and induces cell division or apoptosis depending on the situation (Lee et al., Nat Commun. 2019; 10 (1): RUNX3 regulates cell cycle-dependent chromatin dynamics by functioning as a pioneer factor of the restriction-point). When a K-Ras oncogene mutation occurs in lung epithelial cells, Runx3 kills cancer cells by contributing to determining apoptosis fate at the restriction-point (Lee et al., Nat Commun. 2019; 10 (1))

Runx3 protein refers to a transcription factor related to the Runt family expressed by the Runx3 gene.

A Runx3 protein refers to a Runt-related transcription factor 3 expressed by the Runx3 gene.

The Runx3 protein can be composed of the amino acid sequence represented by SEQ. ID. NO: 1 or SEQ. ID. NO: 2.

The Runx3 protein can be derived from humans or animals.

The Runx3 protein can be synthesized by the conventional chemical synthesis method in the art (W. H. Freeman and Co., Proteins; structures and molecular principles, 1983), or can be prepared by the conventional genetic engineering method (Maniatis et al., Molecular Cloning: A laboratory Manual, Cold Spring Harbor laboratory, 1982; Sambrook et al., Molecular Cloning: A Laboratory Manual et al.).

The Runx3 protein can be a variant of an amino acid sequence having a different sequence by deletion, insertion or substitution of amino acid residues, or a combination thereof within a range that does not affect the function of the protein. Amino acid exchanges in proteins that do not totally alter the activity of the molecule are informed in the art. In some cases, the amino acid can be modified by phosphorylation, sulfation, acrylation, glycosylation, methylation or farnesylation. Accordingly, the present invention can include a peptide having an amino acid sequence substantially identical to that of a protein composed of the amino acid sequence represented by SEQ. ID. NO: 1 or SEQ. ID. NO: 2, and variants or fragments thereof. The substantially identical protein can have homology to the protein of the present invention by 80% or more, particularly 90% or more, and more particularly 95% or more.

The polynucleotide encoding the Runx3 protein can be composed of the nucleotide sequence represented by SEQ. ID. NO: 3 or SEQ. ID. NO: 4.

The polynucleotide encoding the Runx3 protein can be derived from humans or animals.

The vector including the polynucleotide encoding the Runx3 protein can be linear DNA or plasmid DNA.

The vector refers to a transport mediator for introducing the polynucleotide encoding the Runx3 protein of the present invention into a subject to be treated, and can include a promoter suitable for expression in a subject to be treated, an enhancer, and a polynucleotide encoding the Runx3 protein, a transcription termination site, and the like. The promoter can be a specific organ and tissue specific promoter, and can include a replication origin so as to proliferate in the organ and tissue.

The virus transformed by the vector can be any one selected from the group consisting of retrovirus, adenovirus, adeno-associated virus, herpes simplex virus, and lentivirus.

The adeno-associated virus refers to an adeno-associated virus capable of expressing the foreign gene by inserting a target foreign gene, and is also referred to as a recombinant adeno-associated virus vector.

The recombinant adeno-associated virus (rAAV) means, in a narrow sense, an expression vector containing a foreign gene, prepared to allow the expression of the foreign gene in cells infected by the adeno-associated virus, but in a broad sense, the recombinant adeno-associated virus refers to any vector required to be transduced into cells to form a recombinant adeno-associated virus including the following AAV rep-cap gene expression vector and helper plasmid or helper virus.

The AAV rep-cap gene expression vector refers to an expression vector of a gene encoding an enzyme (rep) required for replication of a genome derived from the recombinant adenovirus expression vector and an envelope protein (cap) for formation of adenovirus particles. The expression vector is simultaneously transfected with the recombinant adeno-associated virus expression vector, thereby enabling intracellular production of a recombinant adeno-associated virus. The helper virus refers to a virus that helps to form infectious particles of the adeno-associated virus that cannot replicate independently, and includes adenovirus, vaccinia virus, and herpes simplex virus. The helper plasmid refers to a plasmid that acts on behalf of the helper virus. Meanwhile, the AAV rep-cap gene expression vector and the helper plasmid can be implemented as a single vector, and a representative example is pDG (DKFZ, Germany). Since the AAV rep-cap gene expression vector and the helper virus or the helper plasmid can both help the rAAV expression vector that cannot independently form infectious adeno-associated virus particles to form infectious rAAV particles, in this document, a plasmid (e.g., pDG) simultaneously containing the AAV rep-cap gene and the adenovirus-derived gene necessary for the formation of adeno-associated virus infectious particles is referred to as a helper plasmid, and the helper plasmid and the helper virus are collectively referred to as a helper vector.

In the case of the vector containing the polynucleotide, it is preferably to contain 0.05 to 500 mg, and more preferably to contain 0.1 to 300 mg. In the case of the recombinant virus containing the polynucleotide encoding Runx3 protein, it is preferably to contain 103 to 1012 IU (10 to 1010 PFU), and more preferably to contain 105 to 1010 IU.

The recombinant virus is preferably an adenovirus or an adeno-associated virus, and the number of viruses for treatment can be represented by the number of viral particles including the vector genome or the number of infectable viruses. That is, since about 1% of the virus particles are the effective number of viruses that can actually be infected, IU (infection unit) or PFU (plaque forming unit) is used to indicate this.

The cells transformed by the vector can be bacteria.

The bacteria can be non-pathogenic or non-toxic, and can be Listeria, Shigella, Salmonella, or E. coli. By introducing the vector into the bacteria, DNA of a gene included in the vector can be mass-replicated or proteins can be mass-produced.

The vector according to the present invention can be introduced into cells using a method known in the art. For example, transient transfection, microinjection, transduction, cell fusion, calcium phosphate precipitation, liposome-mediated transfection, DEAE dextran-mediated transfection, polybrene-mediated transfection, electroporation, gene gun, and other known methods for introducing nucleic acids into cells can be used to introduce the vector into cells, but not always limited thereto (Wu et al., J. Bio. Chem., 267:963-967, 1992; Wu and Wu, J. Bio. Chem., 263:14621-14624, 1988).

In the case of the cells transformed with the vector containing the polynucleotide, it is preferably to contain 103 to 10⁸ cells, and more preferably to contain 104 to 10⁷ cells.

The K-Ras mutant lung cancer can be a lung cancer in which the K-Ras mutant gene is activated and the tumor suppressor gene is inactivated.

The tumor suppressor gene can be selected from the group consisting of sPD-1, VHL, MMACI, DCC, p53, NF1, WT1, Rb, BRCA1, BRCA2 and Runx3 genes, but not always limited thereto.

The tumor suppressor gene can be Runx3 gene, but not always limited thereto.

When the activity of Runx3 gene is restored, lung cancer cells are removed and normal cells survive, so that K-Ras mutant lung cancer can be fundamentally cured.

The K-Ras mutant lung cancer can be cured without the possibility of recurrence.

The lung cancer can be non-small cell lung cancer or small cell lung cancer.

The non-small cell lung cancer can be squamous cell carcinoma, large cell carcinoma or lung adenocarcinoma.

The lung adenocarcinoma can be the lung adenocarcinoma induced by the mutation in which glycine (G), the 12^{th} amino acid of K-Ras protein, is substituted with aspartate (D), cysteine (C) or valine (V).

The lung adenocarcinoma can be the lung adenocarcinoma induced by a mutation in which glycine (G), the 13^{th} amino acid of K-Ras protein, is substituted with cysteine (C) or aspartate (D).

The lung adenocarcinoma can be the lung adenocarcinoma induced by a mutation in which alanine (A), the 18^{th} amino acid of K-Ras protein, is substituted with aspartate (D).

The lung adenocarcinoma can be the lung adenocarcinoma induced by a mutation in which glutamine (Q), the 61^{st} amino acid of K-Ras protein, is substituted with histidine (H). The lung adenocarcinoma can be the lung adenocarcinoma induced by a mutation in which lysine (K), the 117^{th} amino acid of K-Ras protein, is substituted with asparagine (N).

The pharmaceutical composition for prevention or treatment of K-Ras mutant lung cancer, comprising a Runx3 (Runt-related transcription factor 3) protein, a polynucleotide coding thereof, a vector carrying the polynucleotide, or a virus or cell transformed with the vector as an active ingredient of the present invention can be administered parenterally during clinical administration.

The effective dose of the composition per 1 kg of body weight is 0.05 to 12.5 mg/kg for the vector, 107 to 1011 virus particles (105 to 109 IU)/kg for the recombinant virus, and 103 to 106 cells/kg for the cell Preferably, the dose is 0.1 to 10 mg/kg for the vector, 108 to 1010 virus particles (106 to 108 IU)/kg for the recombinant virus, and 102 to 10⁵ cells/kg for the cell. The composition can be administered 2 to 3 times a day. The composition as described above is not always limited thereto, and can vary depending on the conditions of a patient and the degree of onset of a disease.

The pharmaceutical composition according to the present invention may contain 10 to 95 weight % of a vector containing a Runx3 protein, a polynucleotide coding thereof, a vector carrying the polynucleotide, or a virus or cell transformed with the vector, which is an active ingredient, based on the total weight of the composition. In addition, the pharmaceutical composition of the present invention can include, in addition to the active ingredient, one or more effective ingredients having the same or similar function to the active ingredient.

The present invention also provides a screening method of a candidate substance for treating K-Ras mutant lung adenocarcinoma comprising the following steps:
1) treating a test substance to the cells containing Runx3 gene;
2) confirming the expression or activity of Runx3 protein in the cells of step 1); and
3) selecting a test substance that increases the expression or activity of Runx3 protein in step 2) compared to the untreated control group.

The expression level of the protein of step 2) can be measured by any one method selected from the group consisting of Western blotting, immunoprecipitation, dual luciferase reporter assay, enzyme-linked immunosorbent assay (ELISA) and immunohistochemistry.

The present invention also provides a method for preventing, ameliorating or treating K-Ras mutant lung cancer comprising a step of administering the Runx3 (Runt-related transcription factor 3) protein, the polynucleotide coding thereof, the vector carrying the polynucleotide, or the virus or cell transformed with the vector to a subject.

The vector according to the present invention can have the characteristics as described above. The subject can be a mammal, specifically a human.

The composition of the present invention can be administered parenterally according to a desired method, and the parenteral administration includes external skin application or intraperitoneal injection, rectal injection, subcutaneous injection, intravenous injection, intramuscular injection, or intrathoracic injection.

The vector of the present invention is administered in a pharmaceutically effective dose. The term “pharmaceutically effective dose” herein indicates the amount enough to treat the disease with applicable, reasonable or risky concentration. The dose can be determined according to the type of disease, the severity, the activity of the drug, the patient's sensitivity to the drug, the time of administration, the route of administration, excretion, the duration of treatment, the drugs being used simultaneously, and other factors regarded as relevant in the medicinal field. The composition of the present invention can be administered alone or in combination with other therapeutic agents. In combination administration, the administration can be sequential or simultaneous. The composition can be administered single or multiple. It is important to administer an amount capable of obtaining a maximum effect in a minimum amount without side effects in consideration of all the above factors, and the amount can be easily determined by a person skilled in the art. A typical dosage unit for determining a therapeutically effective dose is calculated based on the amount of the active ingredient that can be administered to a human subject (70 kg) in a single dose. However, it is understood that the exact therapeutically effective dose of the active ingredient varies with the relative amount of each active ingredient used, the drug used and the rate of elevation.

In addition, the present invention provides a use of the Runx3 (Runt-related transcription factor 3) protein, the polynucleotide coding thereof, the vector carrying the polynucleotide, or the virus or cell transformed with the vector for the manufacture of a medicament for preventing, ameliorating or treating K-Ras mutant lung cancer.

The composition according to the present invention can have the characteristics as described above.

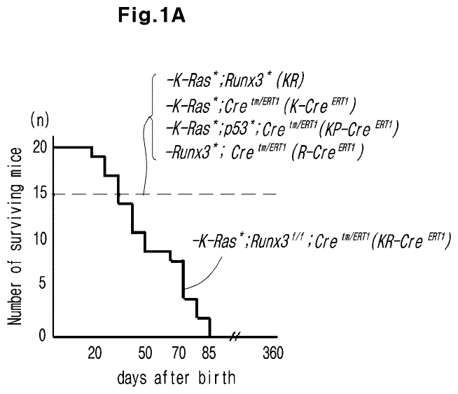

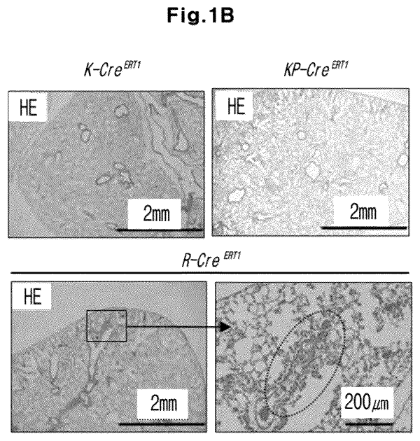

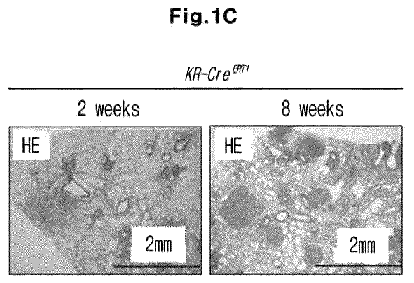

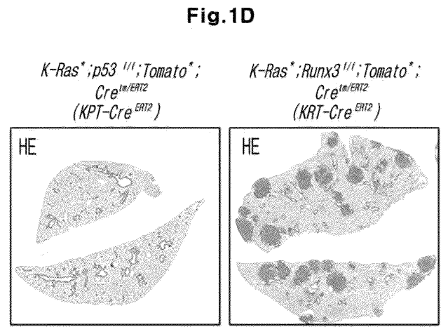

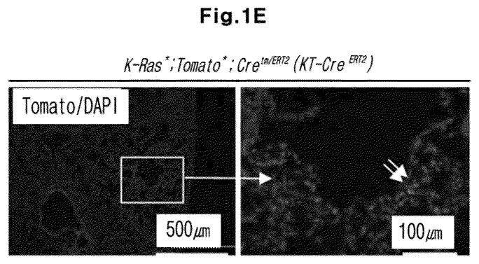

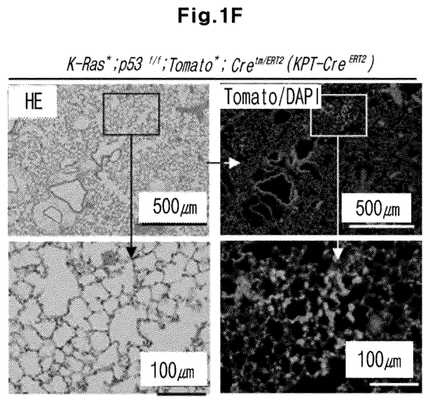

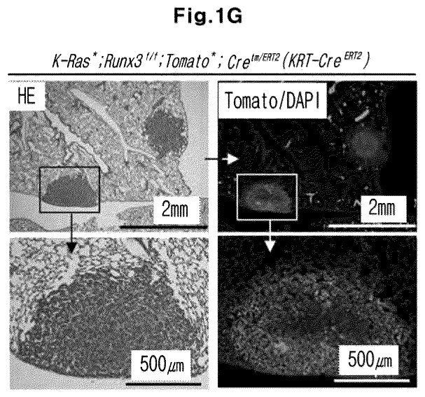

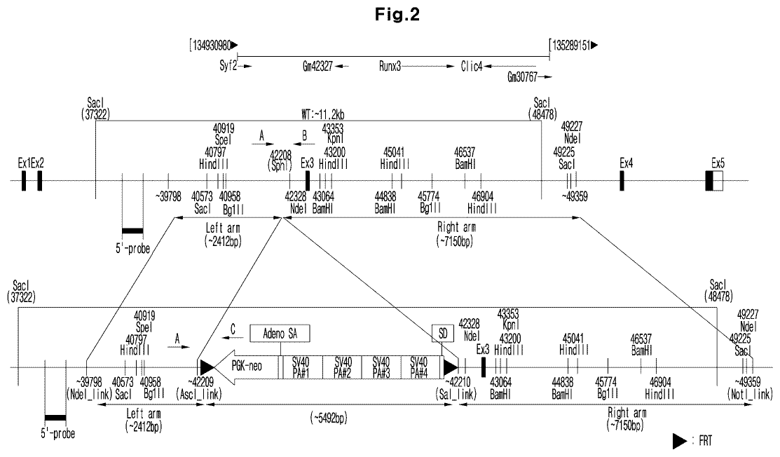

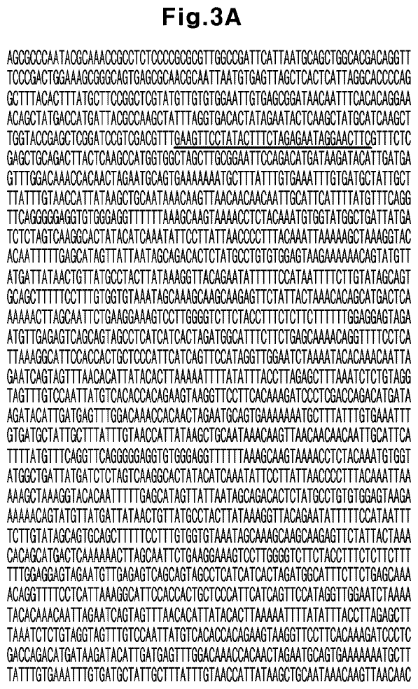

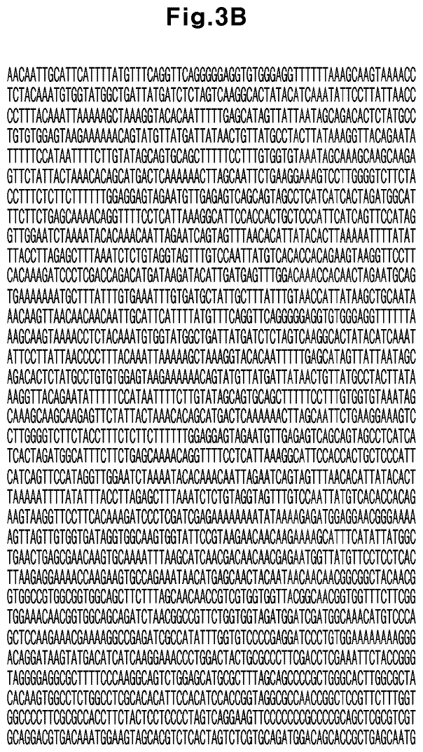

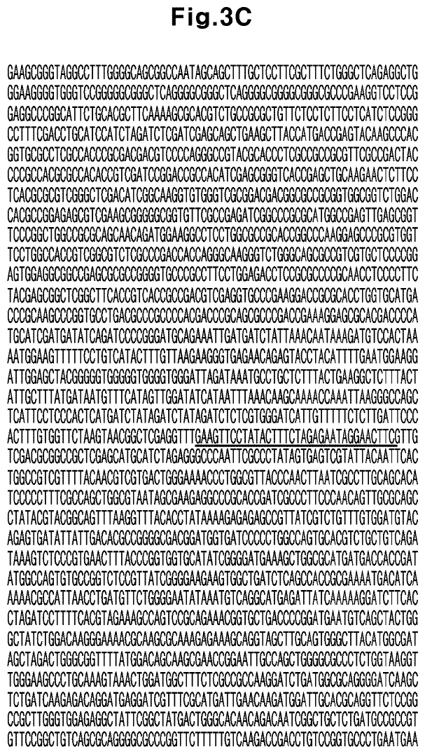

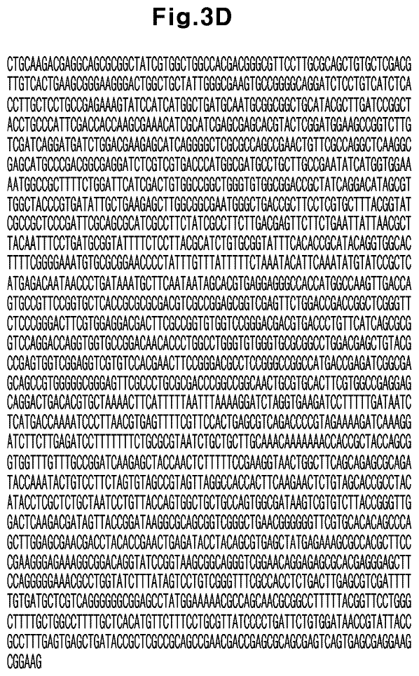

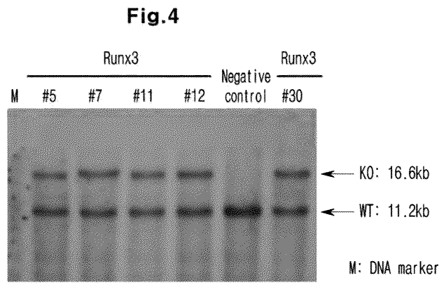

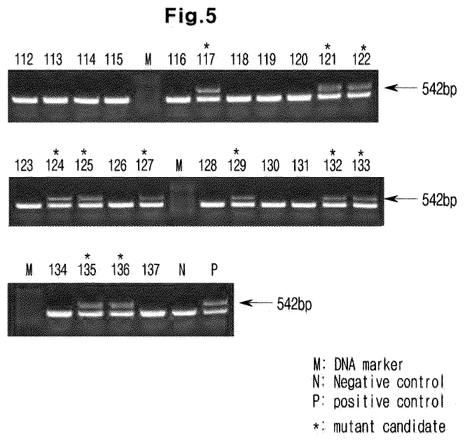

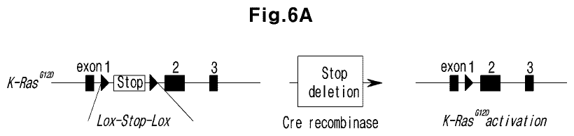

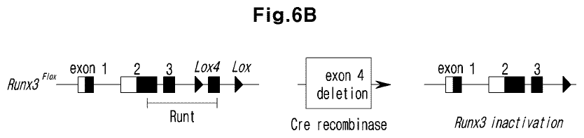

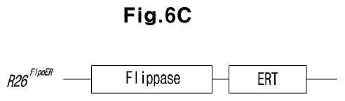

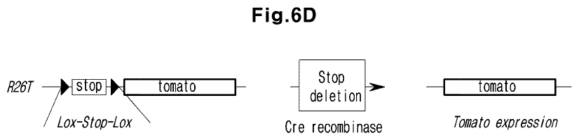

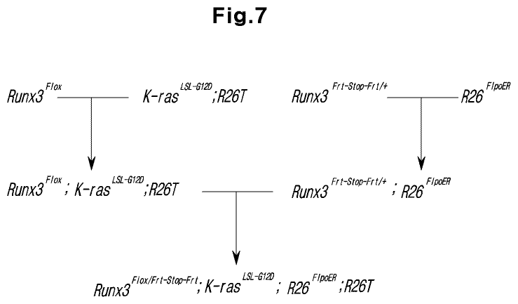

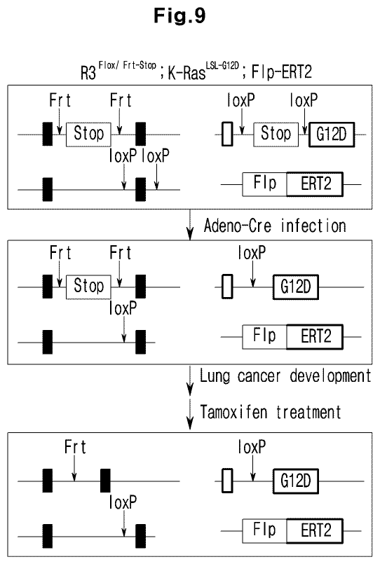

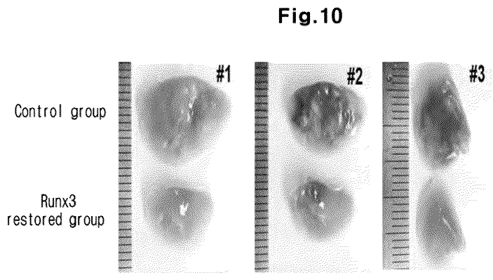

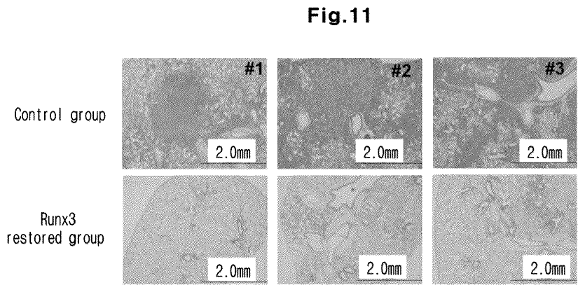

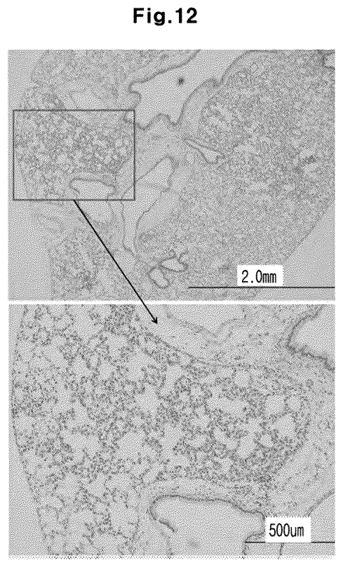

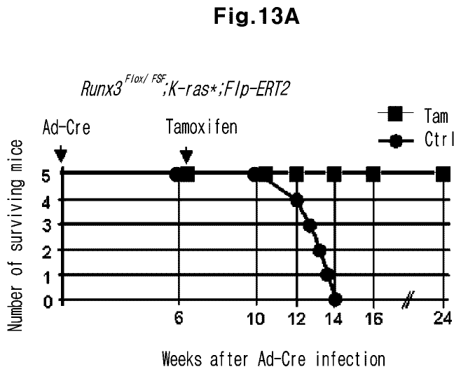

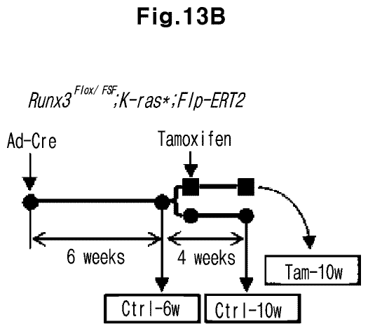

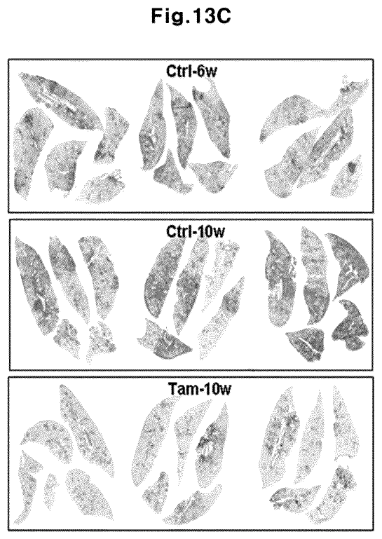

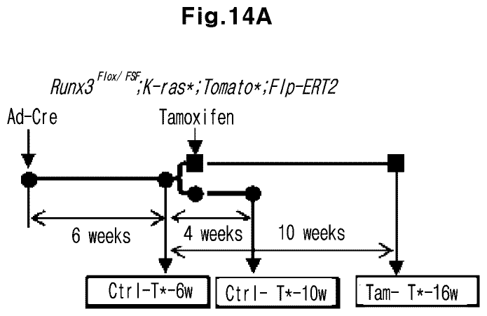

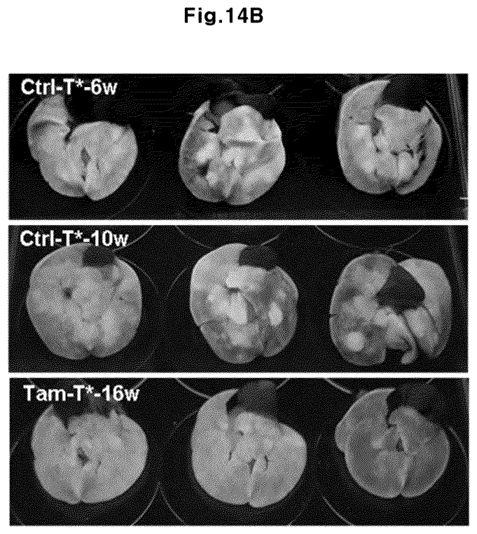

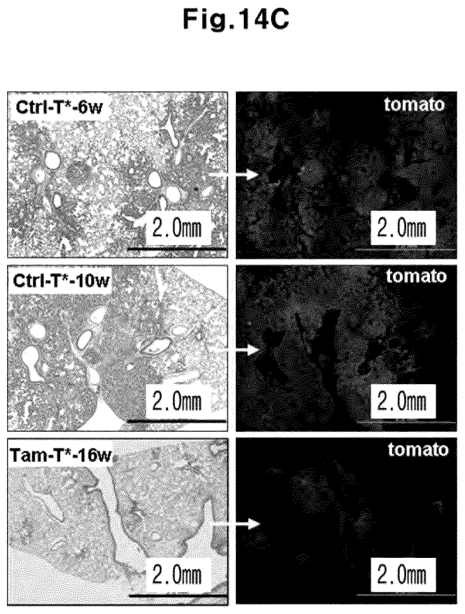

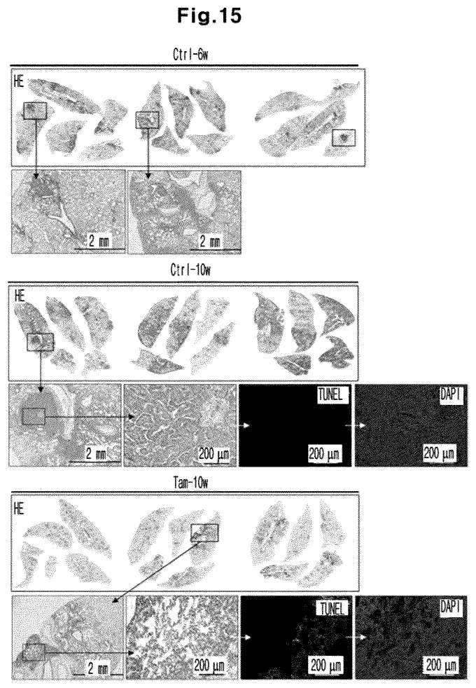

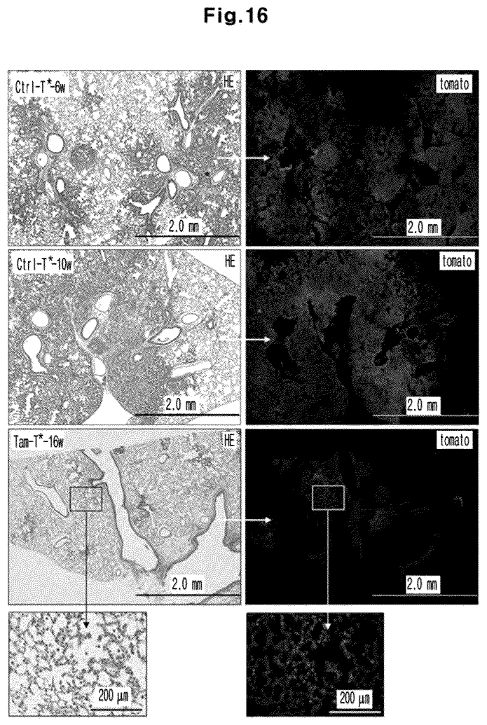

In a preferred embodiment of the present invention, the present inventors confirmed the survival of the mice in which K-Ras gene was inactivated and Runx3 gene was activated, the mice in which K-Ras gene was activated, the mice in which K-Ras gene was activated and p53 gene was suppressed, the mice in which Runx3 gene was suppressed, and the mice in which K-Ras gene was activated and Runx3 gene was suppressed, and the onset of lung cancer therein. As a result, the mice except those with activated K-Ras gene and suppressed Runx3 gene were survived for more than 1 year and no lung cancer was found therein (see FIGS. 1a, 1b, 1d, 1w, and 1f), but all the mice with activated K-Ras gene and suppressed Runx3 gene died within 85 days of birth, and lung cancer was observed therein. Therefore, it was confirmed that lung cancer was developed only when the activation of K-Ras gene and the suppression of Runx3 gene occurred at the same time (see FIGS. 1a, 1c and 1g). Thus, in order to confirm that lung cancer was cured when the function of Runx3 gene suppressed in lung cancer caused by the activation of K-Ras gene and the inactivation of Runx3 gene was restored, a gene targeting vector was constructed by introducing FRT-STOP-FRT cassette that has inactivated Runx3 gene expression but can be repaired by Flippase (see FIGS. 3a to 3d) between exon 2 and exon 3 of Runx3 gene (see FIG. 2), and Runx3^{FRT-STOP-FRT} knock-in mice were prepared by transforming mouse embryonic stem cells with the gene targeting vector (see FIGS. 4 and 5). Then, a mouse model in which lung cancer can occur due to the activation of K-Ras gene and the inactivation of Runx3 gene, but the expression of Runx3 gene can be restored by the introduction of Flippase into the nucleus when tamoxifen is administered later was constructed by mating the five types of mice (Runx3^{FRT-STOP-FRT}, K-Ras^{LSL-G12D}, Runx3^{Flox}, R26^{FlpoER}, and R26T (see FIGS. 6a to 6d) as shown in FIG. 7 (see FIG. 9). As a result of confirming the effect of lung cancer treatment in the constructed mouse model, it was confirmed that lung cancer was almost eliminated in the lung tissue of the mouse group in which Runx3 gene was restored by administration of tamoxifen (see FIGS. 10 and 11). In addition, there was a part of the lung tissue of the Runx3 restored group that looks somewhat abnormal, but the enlarged results confirmed that it was a normal lung tissue (see FIG. 12). In order to further confirm the effect of lung cancer treatment in the mice in which Runx3 gene was restored, a diet containing tamoxifen was fed, and the survival was observed in the mouse group in which Runx3 gene was restored and the control group in which Runx3 gene was inactivated. The control group mice died within 14 weeks of inducing lung cancer by infection with Cre-adenovirus, but the Runx3 restored group mice survived for more than 6 months (see FIG. 13a). Lung cancer incidence was confirmed in the control mice (ctrl-6w) fed with tamoxifen-free diet that were sacrificed 6 weeks after the infection with Cre-adenovirus, the control mice (ctrl-10w) fed with tamoxifen-free diet that were sacrificed 10 weeks after the infection with Cre-adenovirus, and the Runx3 restored group mice (tam-10w) fed with tamoxifen-containing diet for 4 weeks after 6 weeks of the Cre-adenovirus infection (see FIG. 13b). As a result, lung cancer filling the lung tissue was found in ctrl-6w and ctrl-10w, but lung cancer was not observed in tam-10w in which Runx3 gene was restored, confirming that lung cancer was cured (see FIG. 13c). In addition, there was a part of the lung tissue of tam-10w mice that looked somewhat abnormal, but as a result of expanding this part, it was confirmed that this part represented dead cells and was normal alveoli. Therefore, it was confirmed that lung cancer was completely cured by restoring Runx3 gene (see FIG. 15). In order to confirm the effect of lung cancer treatment in the mice in which Runx3 gene was restored in detail, a mouse model expressing the red fluorescent protein tdTomato in lung cancer cells was constructed. Then, the expression of tdTomato protein was observed under ultraviolet light in the lung tissues extracted from the control mice (ctrl-T*-6w) fed with tamoxifen-free diet that were sacrificed 6 weeks after the infection with Cre-adenovirus, the control mice (ctrl-T*-10w) fed with tamoxifen-free diet that were sacrificed 10 weeks after the infection with Cre-adenovirus, and the Runx3 restored group mice (tam-T*-16w) fed with tamoxifen-containing diet for 10 weeks after 6 weeks of the Cre-adenovirus infection (see FIG. 14a). As a result, red fluorescence was observed in the entire lung tissues of ctrl-T*-6w and ctrl-T*-10w mice, but red fluorescence was hardly observed in the lung tissues of tam-T*-16w mice. Therefore, it was confirmed that lung cancer was cured in the Runx3 restored group mice (see FIG. 14b and 14c). There was a part of the lung tissue of tam-10w mice that looked somewhat abnormal, but as a result of expanding this part, it was confirmed that this part was normal alveoli. Thus, it was confirmed that lung cancer was completely cured by restoring Runx3 gene (see FIG. 16).

When the Runx3 activity was restored in lung cancer caused by activation of K-Ras mutant gene and decrease in the activity of Runx3 protein, the lung cancer cells were removed and the normal cells survived. Therefore, lung cancer can be fundamentally cured by administering a Runx3 protein, a polynucleotide coding therefor, a vector carrying the polynucleotide, or a virus or cell transformed with the vector.

Hereinafter, the present invention will be described in detail by the following examples and experimental examples.

However, the following examples and experimental examples are only for illustrating the present invention, and the contents of the present invention are not limited thereto.

### Example 1: Confirmation of Lung Cancer in Mouse Model with Activated K-Ras Gene and Deficient Runx3 Gene

<1-1> Confirmation of Survival of Mouse Model with Activated K-Ras Gene and Deficient Runx3 Gene

In general, cancer is induced by simultaneously expressing K-Ras mutations in tens of millions of cells in animal cancer models caused by K-Ras oncogene, but cancer does not occur when K-Ras oncogene mutations are expressed in a small number of cells. This means that genetic mutations other than K-Ras must be involved in order to develop cancer. Therefore, Cre^{tm/ERT1} mice were used as a method of inducing mutations in very few cells to find other genes that cause cancer other than K-Ras. Cre^{tm/ERT1} mice are the mice in which a gene expressing Cre recombinase by treating tamoxifen was inserted into the chromosome. Cre^{tm/ERT} cannot enter the cell nucleus without tamoxifen, so it cannot express Cre recombinase, but it has been reported that a very small amount of Cre^{tm/ERT1} protein enters the cell nucleus without tamoxifen and causes Cre recombinase activity to cleave the DNA inside the loxP sequence (Kemp, R. et al. Nucleic Acids Res 32, e92, 2004). Therefore, the following experiment was performed to determine whether cancer develops according to the activity or inactivity of K-Ras gene, p53 gene, or Runx3 gene in very few cells of the mouse model.

Particularly, the mice capable of selectively expressing K-RasG12D, an oncogene, by Cre recombinase (K-Ras^{LSL-G12D)}; the mice capable of selectively inhibiting the expression of Runx3 gene by Cre recombinase (Runx3^{Flox}); the mice expressing Cre recombinase by treating tamoxifen, but expressing Cre recombinase in very few cells even without tamoxifen (Cre^{tm/ERT1}), and the mice capable of selectively inhibiting the expression of p53 gene by Cre recombinase (p53^{flox}) were purchased from The Jackson Laboratory (USA) (Table 1).

TABLE 1

| Mouse | Strain | Stock No. |
|---|---|---|
| K-Ras^{LSL-G12D} | B6.129S4-Kras^{tm4Tyj}/J | 008179 |
| Runx3^{Flox} | B6.129P2-Runx3^{tm1Itan}/J | 008773 |
| Cre^{tm/ERT1} | Cg-Tg(CAG-Cre/Esr1)5Amc/j | 004682 |
| p53^{flox} | B6.129P2-Trp53^{tm1Brn}/J | 008462 |

The K-Ras^{LSL-G12D}; Runx3^{Flox} normal mice (KR) in which K-Ras gene was inactivated and Runx3 gene was activated because there was no Cre^{tm/ERT1}, the K-Ras^{LSL-G12D}; Cre^{tm/ERT}mice (K-Cre^{ERT1}) in which Cre recombinase was expressed in very few cells and K-Ras gene was activated in very few cells, the K-Ras^{LSL-G12D};p53^{flox}; Cre^{tm/ERT}mice (KP-Cre^{ERT1}) in which Cre recombinase was expressed in very few cells, so that K-Ras gene was activated in very few cells and p53 gene was suppressed, the Runx3^{Flox}; Cre^{tm/ERT1} mice (R-Cre^{ERT1}) in which Cre recombinase was expressed in very few cells and Runx3 gene was suppressed in very few cells, and the Ras^{LSL-G1 2}D; Runx3^{Flox}; Cre^{tm/ERT1} mice (KR-Cre^{ERT1}) in which Cre recombinase was expressed in very few cells, so that K-Ras gene was activated in very few cells and Runx3 gene was suppressed were prepared by mating the four types of mice, and the survival of the prepared mice was observed.

As a result, the K-Rasl.SL-G120; Runx3^{Flox} mice (KR) in which K-Ras gene was inactivated and Runx3 gene was activated, the K-Ras^{LSL-G12D}; Cre^{tm/ERT}mice (K-Cre^{ERT1}) in which K-Ras gene was activated, the K-Ras^{LSL-G12D}; p53^{flox}; Cre^{tm/ERT1} mice (KP-Cre^{ERT1}) in which K-Ras gene was activated and p53 gene was suppressed, and the Runx3^{Flox}; Cre^{tm/ERT}mice (R-Cre^{ERT1}) in which Runx3 gene was suppressed all survived healthy for more than 1 year (FIG. 1a), and no cancer was found as a result of lung tissue staining (FIG. 1b). In the Runx3Flor; Cre^{tm/ERT1} mice (R-Cre^{ERT1}) in which Runx3 gene was suppressed, a dysmorphic part was observed very rarely, but it was confirmed that cancer did not develop. In addition, it was confirmed in KP-Cre^{ERT1} mice that the destruction of p53 did not promote cancer development by the K-Ras mutant oncogene. On the other hand, in the K-Ras^{LSL-G12D}; Runx3^{Flox}; Cretin/ERT¹ mice (KR-Cre^{ERT1}) in which K-Ras gene was activated and Runx3 gene was suppressed, lung adenocarcinoma rapidly developed from 14 days after birth, and all the mice died within 85 days after birth, and cancer filling the lung tissue was observed (see FIGS. 1a and 1c). Through the above results, it was confirmed that fatal disease occurred only in the mice in which K-Ras gene was activated and Runx3 gene was suppressed, and that Runx3 strongly suppressed the development of lung cancer caused by K-Ras mutation.

<1-2> Confirmation of Cancer in Mouse Model with Activated K-Ras Gene and Deficient Runx3 Gene

In order to further clarify the results confirmed in Example <1-1>, the Creim ERT2 mice exhibiting Cre recombinase activity in significantly fewer cells than Cre^{tm/ERT1} in the absence of tamoxifen, the mice (R26T) capable of selectively expressing Rosa26R-Tomato gene and showing red fluorescence by expressing the red fluorescent protein tdTomato by Cre recombinase, and the K-Ras^{LSL-G12D}, Runx3^{Flox} and p53^{flox} mice of Table 1 (Table 2).

TABLE 2

| Mouse | Strain | Stock No. |
|---|---|---|
| Cre^{tm/ERT2} | Gt(ROSA)26Sor^{tm1(Cre/ERT2)Tyj}/J | 008463 |
| R26T | B6.Cg-Gt(ROSA) | 007914 |
| | 26Sor^{tm14(CAG-tdTomato)Hze}/J | |

Particularly, cancer incidence was observed by H&E staining and the expression of the red fluorescent protein tdTomato in the K-Ras^{LSL-G12D}; R26T; Cremm/ERT2 mice (KT-Cre^{ERT2}) in which K-Ras gene was activated in very few cells, the K-Ras^{LSL-G12D};p53^{flox}; R26T;Cre^{tm/ERT2} mice (KPT-Cre^{ERT2}) in which K-Ras gene was activated in very few cells and p53 gene was suppressed, and the K-Ras^{LSL-G12D}; Runx3^{Flox}; R26T; Cre^{tm/ERT2} mice (KRT-Cre^{ERT2}) in which K-Ras gene was activated in very few cells and Runx3 gene was suppressed constructed by mating the five types of mice.

As a result, cancer was not observed in the K-Ras^{LSL-G12D}; R26T; Creim ERT2 mice (KT-Cre^{ERT2}) and the K-Ras^{LSL-G12D};p53^{flox}; R26T; Cre^{tm/ERT2} mice (KPT-Cre^{ERT2}), but cancer filling the lung tissue was observed in the K-Ras^{LSL-G12D}; Runx3^{Flox}; R26T; Cre^{tm/ERT2} mice (KRT-Cre^{ERT2}) (FIG. 1d). In addition, Tomato-positive cells were rarely observed in the K-Ras^{LSL-G1 2}D;R26T;Cre^{tm/ERT2} mice (KT-Cre^{ERT2}) (FIG. 1e), and a part that looked somewhat abnormal was observed in the K-Ras^{LSL-G12D};p53flex; R26T; Creim ERT2 mice (KPT-Cre^{ERT2}), but as a result of expanding this part, it was confirmed that this part was normal alveoli (FIG. 1f). On the other hand, cancer filling the lung tissue was found in the K-Ras^{LSL-G12D}; Runx3^{Flox};R26T;Cre^{tm/ERT}2 mice (KRT-Cre^{ERT2}) (FIG. 1g). From the above results, it was reconfirmed that the gene that suppresses the occurrence of lung cancer caused by the mutation of K-Ras oncogene was Runx3, not p53.

From the results of Examples <1-1> and <1-2>, it was confirmed that cancer did not occur when K-Ras gene was activated alone or when K-Ras gene was activated and p53 gene was suppressed, but it was confirmed that lung cancer was developed when K-Ras activation and Runx3 gene suppression occurred at the same time. The above results indicates that K-Ras mutant lung cancer does not occur with the mutation of K-Ras alone, but cancer occurs only when the suppression of the cancer suppressor gene occurs simultaneously, and that cancer does not occur when p53 gene is suppressed among cancer suppressor genes, but cancer occurs only when Runx3 gene is suppressed at the same time.

Therefore, to confirm whether lung cancer was cured by restoring the activity of the suppressed Runx3 gene when K-Ras activation and Runx3 suppression occurred at the same time, the following experiment was performed.

### Example 2: Construction of Runx3^{FRT-STOP-FRT} Knock-In Mice

A mouse model having a Runx3 allele in which Runx3 gene expression was inactivated but can be repaired by Flippase and FRT (Flippase Recognition Target)-STOP-FRT cassette was introduced between exon 2 and exon 3 of an allele of Runx3 gene was constructed as follows.

### <2-1> Construction of Gene Targeting Vector

A gene targeting vector was constructed by introducing FRT-STOP-FRT cassette between exon 2 and exon 3 of Runx3 gene.

Particularly, by analyzing the Runx3 gene sequence, a vector was designed so that FRT-STOP-FRT cassette (SEQ. ID. NO: 5) was introduced into the second SphI restriction enzyme site in the 5′-intron direction of exon 3 of Runx3 gene. The Runx3 gene sequence was obtained from NCBI database. The FRT-STOP-FRT cassette was introduced into the SphI restriction enzyme site (GCATGC), which is the 42208^{th} nucleotide from the first nucleotide of the nucleotide sequence at NC_000070.6 site of mouse chromosome #4 (Mus musculus Runx3-Chromosome4-NC_000070.6; 135120645˜135177990). The FRT-STOP-SRT cassette was purchased from Addgene (USA) (Fret-stop-Fret TOPO plasmid, Cat #. 22774) and used (FIGS. 2 and 3).

### <2-2> Construction of Gene-Targeted Embryonic Stem Cells

A transgenic mouse construction service was requested from Macrogen (Korea). The vector prepared in Example <1-1> was transformed into mouse embryonic stem cells, and southern blotting was performed to select embryonic stem cells in which homologous recombination occurred.

Particularly, the FRT-STOP-SRT cassette introduced gene targeting vector was linearized by digesting it with SacI, and then transformed into mouse embryonic stem cells by electroporation. After the transformation, genomic DNA (gDNA) was extracted from a total of 30 groups of embryonic stem cells first selected, digested with SacI restriction enzyme, and then Southern blotting was performed using the 5′-probe shown in FIG. 2. The 5′-probe was used as an 1157 bp long DNA fragment made by digesting the DNA with SacI and EcoRI restriction enzymes. When performing Southern blotting, DNA of normal cells was used as the negative control.

As a result of Southern blotting, bands were found at both 11.2 kb (wild-type, WT) and 16.6 kb (mutant, KO) sites in the 5 groups (5, 7, 11, 12, and 30) of a total of 30 groups of embryonic stem cell samples. Therefore, embryonic stem cells of the 5 groups were selected as gene-targeted embryonic stem cells with homologous recombination (FIG. 4).

### <2-3> Construction of Runx3^{FRT-STOP-FRT} Knock-In Mice

Chimeric mice were produced using the gene-targeted embryonic stem cells selected in Example <2-2> through the transgenic mouse construction service of Macrogen (Korea). F1 generation mice were produced therefrom, and then Runx3^{FRT-STOP-FRT} knock-in mice into which FRT-STOP-FRT cassette was introduced were selected by performing polymerase chain reaction (PCR).

Particularly, the gene-targeted embryonic stem cells were injected into a blastocyst of a FVB mouse, and then transplanted into a surrogate mother to produce a chimeric mouse. Genomic DNA (gDNA) was extracted from the tail of a baby mouse (F1) born by crossing the chimeric mouse and the FVB line wild-type mouse. Then, PCR was performed according to the conditions shown in Table 4 using the primers of Table 3 below that can complementarily bind to the positions shown in FIG. 2 (A to C).

TABLE 3

| | | SEQ. |
|---|---|---|
| Primer | Sequence (5′→3′) | ID. NO: |
| Runx3-SC- | CTGTGTAGTCCTGGCTATCC | 6 |
| F1(forward)_A | T | |
| Runx3-SC- | CTTAGCTGTCCTCCGACTAC | 7 |
| R1(reverse)_B | A | |
| OS-Neo-F1 | GGATGATCTGGACGAAGAGC | 8 |
| (reverse)_C | A | |

TABLE 4

| Temperature | | |
|---|---|---|
| (° C.) | Time | Cycle |
| 94 | 5 minutes | 1 |
| 94 | 30 seconds | 35 |
| 60 | 30 seconds | |
| 72 | 30 seconds | |
| 72 | 10 minutes | 1 |

| | |
|---|---|
| 4 | — |

As a result, bands were found at 542 bp site in genes of 11 mice out of a total of 26° F.1 generation mice. Therefore, these mice were finally selected as Runx3TRT-STOP-FRT knock-in mice (Runx3FSF mice) (FIG. 5).

### Example 3: Construction of Runx3^{Flox}/FRT-STOP-FRT. K-Rast.SL-G12D. R26^{FlpoER}. R26T mice

In order to verify the therapeutic effect of restoring Runx3 gene on K-Ras mutant lung adenocarcinoma, each gene recombinant mouse was purchased and crossed to construct the mice in which Runx3 gene expression is inactivated but can be restored by Flippase.

Particularly, the mice capable of selectively expressing the oncogene K-Ras^{G12D} using Cre recombinase (FIG. 6a), the mice capable of selectively inhibiting the expression of Runx3 gene using Cre recombinase (FIG. 6b), the mice capable of introducing Flippase, which is fused with a modified estrogen receptor (ERT), into the cell nucleus when treated with tamoxifen, an estrogen analogue (FIG. 6c), and the mice capable of selectively expressing tdTomato, a red fluorescent protein, using Cre recombinase (FIG. 6d) were purchased from the Jackson Laboratory (USA) (Table 5).

TABLE 5

| Mouse | Strain | Stock No. |
|---|---|---|
| K-Ras^{LSL-G12D} | B6.129S4-Kras^{tm4Tyj}/J | 008179 |
| Runx3^{Flox} | B6.129P2-Runx3^{tm1Itan}/J | 008773 |
| R26^{FlpoER} | B6N.129S6(Cg)- | 019016 |
| | Gt(ROSA) | |
| | 26Sor^{tm3(CAG-flpo/ERT2)Alj}/J | |
| R26T | B6.Cg-Gt(ROSA) | 007914 |
| | 26Sor^{tm14(CAG-tdTomato)Hze}/J | |

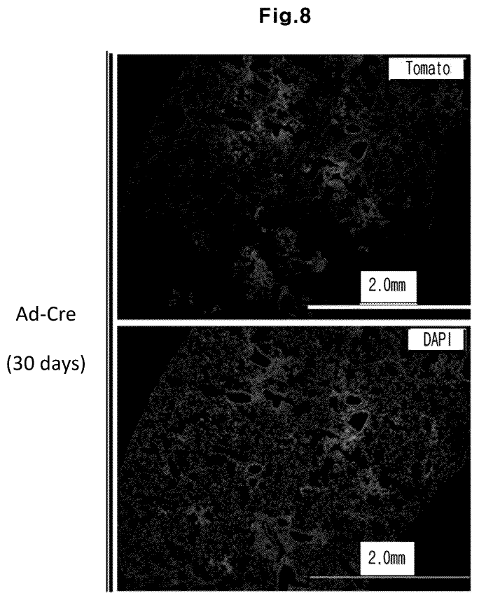

K-Ras^{LSL-G12D} mice were crossed with R26T mice to construct K-Ras^{LSL-G12D};R26T mice, and these mice and the Runx3^{FRT-STOP-FRT} mice prepared in Example 2 were crossed as shown in FIG. 7 to finally construct Runx3^{Flox} TRT-STOP-FRT; K-Ras^{LSL-G12D}; R26^{FlpoER}; R26T mice. Immunofluorescence staining was performed on lung cancer cells of the constructed mice. As a result, it was confirmed that the red fluorescent protein tdTomato was expressed, indicating that the selective genetic manipulation was normally performed (FIG. 8). In addition, Runx3^{Flox/FRT-STOP-FRT};K-Ras^{LSL-G12D}; R26^{FlpoER} mice were also constructed in the same manner as shown in FIG. 7 except that the process of crossing K-Ras^{LSL-G12D} mice with R26T mice was omitted.

### Experimental Example 1: Confirmation of Cancer Treatment Effect by Restoring Runx3 Gene on K-Ras Mutant Lung Adenocarcinoma Mice Lacking Runx3 Gene

<1-1> Construction of K-Ras Mutant Lung Adenocarcinoma Mice with Inactivated Runx3 Gene

The Runx3^{Flox/FRT-STOP-FRT}; K-Ras^{LSL-G12D}; R26^{FlpoER}; R26T mice (8 weeks old) constructed in Example 3 were respiratory infected with the adenovirus expressing Cre recombinase (Cat. No. 1045, Vector Biolabs, USA) through the nose, so that the Runx3 gene expression was selectively suppressed in only lung cells and K-RasG12D was expressed (FIG. 9). Six weeks after the infection, lung adenocarcinoma was visually confirmed.

### <1-2> Confirmation of Cancer Treatment Effect by Restoring Runx3 Gene on K-Ras Mutant Lung Adenocarcinoma Mice (1)

When tamoxifen is administered to the lung adenocarcinoma mice of Experimental Example <1-1>, Flippase can enter the nucleus and remove the STOP sequence in the FRT-STOP-FRT cassette, so that the suppressed Runx3 gene can be re-expressed (FIG. 9). Accordingly, the mice of Experimental Example <1-1> were fed a feed containing 400 mg/kg of tamoxifen 6 weeks after the adenovirus infection to restore Runx3 gene to a normal state. The feed containing tamoxifen (Cat. #. T5648) was a custom feed (Teklad Custom Diet, TD.130860) of Envigo (UK) produced by Doo Yeol Biotech. (Korea). After feeding the tamoxifen-containing feed for 4 weeks, the control mice fed with the tamoxifen-free feed and the Runx3 restored group mice fed with the tamoxifen-containing feed were sacrificed and the lung tissues were extracted therefrom. Then, the presence of lung adenocarcinoma and the lung tissue size were observed.

As a result, large lung adenocarcinoma was observed in the control group, whereas lung adenocarcinoma was almost removed from the lung tissue of the Runx3 restored group mice, and the size was remarkably small (FIG. 10). The above results suggest that K-Ras mutant lung adenocarcinoma can be treated by restoring Runx3. Tamoxifen was administered for the purpose of restoring Runx3 gene. It is well known that the tamoxifen itself has no anticancer effect on the treatment of lung cancer caused by mutations of K-Ras oncogene (Feldser, D. M. et al., Nature, 468:572-575, 2010, Junttila, M. R. et al., Nature, 468:567-571, 2010). Therefore, it was confirmed that the lung cancer treatment effect was not due to the anticancer effect of the tamoxifen itself, but because the Flippase activated by the administration of tamoxifen restored Runx3.

### <1-3> Confirmation of Cancer Treatment Effect by Restoring Runx3 Gene on K-Ras Mutant Lung Adenocarcinoma Mice (2)

H&E (hematoxylin & eosin) staining was performed using the mouse lung tissues extracted in Experimental Example <1-2>.

Particularly, the extracted mouse lung tissue was fixed in 10% formalin solution for 24 hours, and then paraffin was infiltrated into the tissue using a tissue processor (Leica, Germany). This was manufactured as a paraffin block, and then prepared into 5 μm-thick sections (Leica). The prepared tissue section was attached to a slide glass and dried in an oven at 60° C. for 1 hour. The slide glass was left in xylene 4 times for 5 minutes each, in 100% ethanol for 1 minute, in 95% ethanol for 3 minutes, in 80% ethanol for 3 minutes, and in 70% ethanol for 3 minutes. Paraffin was removed from the tissue section by washing three times with distilled water for 5 minutes each. Then, the slide glass was immersed in a hematoxylin solution for 5 minutes to stain the cell nuclei in blue, and washed with flowing distilled water. The slide glass was immersed in an eosin solution for 1 minute to stain the cytoplasm in red, and washed with flowing distilled water. The stained tissue was observed by taking photographs with a microscope.

As a result, lung cancer filling the lung tissue was observed in the control group mice, whereas no cancer was observed in the lung tissue of the Runx3 restored group mice (FIG. 11). A part that looked somewhat abnormal was observed in the lung tissue of the Runx3 restored mice, but it was confirmed that this part represented traces of cancer formation and treatment and was not the abnormal tissue (FIG. 12). In addition, the Runx3 restored group mice did not show side effects such as weight loss. The above results suggest that K-Ras mutant lung adenocarcinoma can be treated by restoring Runx3.

### <1-4> Confirmation of Cancer Treatment Effect by Restoring Runx3 Gene on K-Ras Mutant Lung Adenocarcinoma Mice (3)

In order to observe the cancer growth in the 3^{Flox/FRT-STOP-FRT};K-Ras^{LSL-G12D}; R26^{FlpoER} mice produced in Example 2, the lung tissues were extracted from the control mice (ctrl-6w) that were sacrificed 6 weeks after infecting with Cre-adenovirus and feeding with tamoxifen-free diet, the control mice (ctrl-10w) that were sacrificed 10 weeks after infecting with Cre-adenovirus and feeding with tamoxifen-free diet, and the Runx3 restored group mice (tam-10w) fed with tamoxifen-containing diet for 4 weeks after 6 weeks of the Cre-adenovirus infection, and H&E (hematoxylin & eosin) staining and TUNEL (terminal deoxynucleotidyl transferase dUTP nick-end labeling) staining were performed (FIG. 13b).

Particularly, H&E staining was performed in the same manner as described in Experimental Example <1-3>, and TUNEL staining, a method of identifying a place where DNA is broken during apoptosis by fluorescent labeling, was performed as follows. The mouse lung tissue was inflated in 4% paraformaldehyde or 3.7% formaldehyde and fixed for 36 hours, and then paraffin was infiltrated. The tissue was made into a paraffin block, and the fixed paraffin section was attached to a slide glass and dried in an oven at 60° C. for 1 hour. The slide glass was left in xylene 4 times for 5 minutes each, rehydrated through an alcohol gradient, and 0.02 mg/ml of Proteinase K solution was treated to allow the staining reagent to penetrate into DNA. After that, TUNEL staining was performed using a kit by the method provided by Roche. Then, the TUNEL-stained tissues were observed under a microscope.

As a result, the control mice survived for 10 weeks after the infection with Cre-adenovirus, and all died after 14 weeks. On the other hand, the Runx3 restored group mice fed with tamoxifen-containing diet 6 weeks after the Cre-adenovirus infection survived until 24 weeks after the infection (FIG. 13a). In the control mice (ctrl-6w) that were sacrificed 6 weeks after infecting with Cre-adenovirus and feeding with tamoxifen-free diet, lung cancer filling about half of the heavily stained lung tissue was observed. In the control mice (ctrl-10w) that were sacrificed 10 weeks after infecting with Cre-adenovirus and feeding with tamoxifen-free diet, lung cancer filling more heavily stained lung tissue than in the ctrl-6w group was observed. On the other hand, no cancer was observed in the lung tissue of the Runx3 restored group mice (FIG. 13c). A part that looked somewhat abnormal was observed in the lung tissue of the Runx3 restored mice (tam-10w), but it was confirmed that this part represented traces of cancer formation and treatment. This part was seen as TUNEL-positive green, so it represented dead cells. As a result of DAPI staining, it was confirmed as normal alveoli, the normal tissue (Figure 15). The above results suggest that K-Ras mutant lung adenocarcinoma can be treated by restoring Runx3.

### <1-5> Confirmation of Cancer Treatment Effect by Restoring Runx3 Gene on K-Ras Mutant Lung Adenocarcinoma Mice (4)

To observe the long-term effect of Runx3 recovery and the progression of cancer cells in lung cancer, the Runx3^{Flox/FRT-STOP-FRT};K-Ras^{LSL-G12D}; R26^{FlpoER};R26T mice constructed in Example 2 were respiratory infected with the adenovirus expressing Cre recombinase as shown in Experimental Example <1-1>.

The lung tissues were extracted from the control mice (ctrl-T*-6w) fed with tamoxifen-free diet that were sacrificed 6 weeks after the infection with Cre-adenovirus, the control mice (ctrl-T*-10w) fed with tamoxifen-free diet that were sacrificed 10 weeks after the infection with Cre-adenovirus, and the Runx3 restored group mice (tam-T*-16w) fed with tamoxifen-containing diet for 10 weeks after 6 weeks of the Cre-adenovirus infection, and observed under ultraviolet light. H&E staining was performed in the same manner as described in Experimental Example <1-3>, and the stained tissues were observed under a microscope, and the expression of tdTomato, a red fluorescent protein, was also observed under a microscope (FIG. 14a).

As a result, about half of tdTomato was expressed and lung cancer filling about half of the lung tissue was observed in ctrl-T*-6w mice, and more tdTomato was expressed and lung cancer filling more lung tissue than ctrl-T*-6w was observed in ctrl-T*-10w mice. On the other hand, no cancer was observed in the lung tissue of the Runx3 restored group mice (tam-T*-16w) (FIG. 14b). The lung tissues were observed under a microscope. As a result, medium-sized cancer cells were observed in ctrl-T*-6w mice, and very large cancer cells were observed in ctrl-T*-10w mice. On the other hand, fluorescence by tdTomato was hardly observed in the Runx3 restored group mice (tam-T*-16w) (FIG. 14c) As shown in FIG. 16 magnifying the microscopic observation photograph, some red fluorescence was observed in the lung tissue of the Runx3 restored group mice (tam-T*-16w). However, this was not the dimorphic tissue but the normal alveoli, so it was confirmed that the cancer cells were completely removed. Therefore, the above results suggest that K-ras mutant lung cancer can be completely cured without the possibility of recurrence by restoring Runx3.

1. A method of treating K-Ras mutant lung cancer in a subject, comprising administering a polynucleotide coding a Runx3 (Runt-related transcription factor 3) protein, a vector carrying the polynucleotide, or a virus or cell transformed with the vector, in a pharmaceutically effective amount to the subject.

2. The method according to claim 1, wherein the Runx3 protein is composed of the amino acid sequence represented by SEQ ID NO: 1 or SEQ ID NO: 2.

3. The method according to claim 1, wherein the polynucleotide coding the Runx3 protein is composed of the nucleotide sequence represented by SEQ ID NO: 3 or SEQ ID NO: 4.

4. The method according to claim 1, wherein the Runx3 protein is a variant of an amino acid sequence having a different sequence by deletion, insertion or substitution of amino acid residues, or a combination thereof within a range that does not affect the function of the protein.

5. The method according to claim 1, wherein the Runx3 protein has 95% or more homology with the amino acid sequence represented by SEQ ID NO: 1 or SEQ ID NO: 2.

6. The method according to claim 1, wherein the vector is linear DNA or plasmid DNA.

7. The method according to claim 1, wherein the virus is any one selected from the group consisting of retrovirus, adenovirus, adeno-associated virus, herpes simplex virus, and lentivirus.

8. The method according to claim 1, wherein the cell is bacteria.

9. The method according to claim 1, wherein the K-Ras mutant lung cancer is lung cancer in which a K-Ras mutant gene is activated and a Runx3 gene is inactivated.

10. The method according to claim 9, wherein when the activity of the Runx3 gene is restored, lung cancer cells are removed and normal cells survive, and the K-Ras mutant lung cancer is fundamentally cured.

11. The method according to claim 10, wherein the K-Ras mutant lung cancer is cured without the possibility of recurrence.

12. The method according to claim 9, wherein the lung cancer is non-small cell lung cancer or small cell lung cancer.

13. The method according to claim 12, wherein the non-small cell lung cancer is squamous cell carcinoma, large cell carcinoma, or lung adenocarcinoma.

14. The method according to claim 13, wherein the lung adenocarcinoma is lung adenocarcinoma induced by a mutation in which glycine (G), the 12^{th} amino acid of K-Ras protein, is substituted with aspartate (D), cysteine (C) or valine (V).

15. The method according to claim 13, wherein the lung adenocarcinoma is lung adenocarcinoma induced by a mutation in which glycine (G), the 13^{th} amino acid of K-Ras protein, is substituted with cysteine (C) or aspartate (D).

16. The method according to claim 13, wherein the lung adenocarcinoma is lung adenocarcinoma induced by a mutation in which alanine (A), the 18^{th} amino acid of K-Ras protein, is substituted with aspartate (D).

17. The method according to claim 13, wherein the lung adenocarcinoma is lung adenocarcinoma induced by a mutation in which glutamine (Q), the 61st amino acid of K-Ras protein, is substituted with histidine (H).

18. The method according to claim 13, wherein the lung adenocarcinoma is lung adenocarcinoma induced by a mutation in which lysine (K), the 117^{th} amino acid of K-Ras protein, is substituted with asparagine (N).

19. A screening method of a candidate substance for treating K-Ras mutant lung cancer comprising the following steps:
(i) treating a test substance to the cells containing Runx3 gene;
(ii) confirming the expression or activity of Runx3 protein in the cells of step i); and
(iii) selecting a test substance that increases the expression or activity of Runx3 protein in step ii) compared to the untreated control group.

20. The method of claim 1, wherein:
(i) the Runx3 protein comprises an amino acid sequence of SEQ ID NO: 1,
(ii) the vector is an adeno-associated vector, and
(iii) the K-Ras mutant lung cancer is a lung adenocarcinoma induced by a mutation in which glycine (G), the 12^{th} amino acid of the K-Ras protein, is substituted with aspartate (D).
